(19) United States
(12) Patent Application Publication
(10) Pub. No.: US 2015/0157742 A1
(43) Pub. Date: Jun. 11, 2015

(54) SYNTHESIS OF BIOLOGICAL COMPOUNDS LABELED WITH THE ALPHA EMITTER Ac-225

(71) Applicant: The European Atomic Energy Community (EURATOM), represented by the European Commission, Brussels (BE)

(72) Inventors: Alfred MORGENSTERN, Karlsruhe (DE); Frank BRUCHERTSEIFER, Karlsruhe (DE); Christos APOSTOLIDIS, Heidelberg (DE)

(73) Assignee: The European Atomic Energy Community (EURATOM), represented by the European Commission, Brussels (BE)

(21) Appl. No.: 14/102,865

(22) Filed: Dec. 11, 2013

(51) Int. Cl. A61K 51/00

(52) U.S. Cl. CPC A61K 51/00

(57) ABSTRACT

A method is described for producing a radioconjugate labeled with radionuclide Ac-225 comprising the step of chelating said radionuclide Ac-225 with a conjugated chelate compound in a chelation reaction mixture to obtain a radioconjugate labeled with Ac-225, wherein the pH of the chelation reaction mixture is comprised between 7.1 and 10, preferably between 8.0 and 9.5, more preferably about 9.0.

### TECHNICAL FIELD

The present invention generally relates to an improved protocol for the synthesis of biological compounds labelled with the alpha emitter Ac-225.

### BACKGROUND ART

Radiotherapeutic treatment of cellular disorders, including cancer and infectious diseases is widely documented in literature. A variety of methods have been developed in order to utilise radionuclides in radiotherapy, including targeted radiotherapy, pre-targeted radiotherapy and the use of radionuclides in the form of bone-seeking complexes.

Targeted alpha therapy is a site directed treatment modality for cellular disorders, including cancer and infectious diseases, using alpha radiation to selectively destroy targeted cells, e.g. tumour cells, fungal cells or bacteria. The principle of targeted alpha therapy is based on the coupling of alpha-emitting radionuclides to targeting moieties, e.g. monoclonal antibodies or peptides, that recognise a structure in, on or near a target. Due to the short radiation path length of alpha particles in human tissue (<100 μm), targeted alpha therapy has the potential of delivering a highly cytotoxic radiation dose to targeted cells, while limiting the damage to surrounding healthy tissue.

It is well known within the art that radioconjugates are of high interest for clinical applications. In fact, those compounds are advantageous for therapeutic and diagnostic applications because they are complexed with radioactive metal ions. These types of complexes may e.g. be used to carry radioactive metals to tumour cells which may be targeted for example by the specificity of an attached antibody.

Although a number of methods to synthesise radioconjugates are known, they generally are subject to some drawbacks, either because they require multiple preparation steps in the presence of the radionuclide and/or because the preparation times are long and/or because the yields obtained in terms of radioconjugate are modest.

### Technical Problem

It is an object of the present invention to provide an improved method for preparing radioconjugates, in particular radioconjugates labeled or chelated with actinium-225 (Ac-225). This improved method should allow for a more efficient and fast manufacture of radioconjugates useful in diagnostic and clinical applications.

### General Description of the Invention

Hence, in order to overcome the above-mentioned drawbacks of the existing methods, the present invention proposes a method for producing a radioconjugate labeled with radionuclide actinium-225 (Ac-225) comprising the following step of:
(C) chelating radionuclide Ac-225 with a conjugated chelate compound in a chelation reaction mixture to obtain a radioconjugate labeled with Ac-225,
   wherein the pH of the chelation reaction mixture is comprised between 7.1 and 10, preferably between 8.0 and 9.5, more preferably about 9.0.

It has indeed been surprisingly found that when working at relatively basic to fairly basic pH values, the chelating reaction kinetics is significantly improved. As a consequence, the major advantage of the present method with respect to known methods is that the operation within the particular pH range indicated above not only drastically reduces the reaction times needed, but at the same time allows for high yields of radioconjugate. Generally, the chelating reaction yields obtained are well above 80%, often even above 90% of the initial reactants, although the reaction time is less than a tenth or less of that of comparable methods. A further advantage of the method is that the operation within the temperature range indicated allows for synthesis of radioimmuno-conjugates containing heat sensitive biological compounds such as antibodies or fragments thereof. The present method therefore represents an easy, efficient and useful one-step express chelation process for the preparation of radio(immuno)-conjugates.

Hence, the chelation reaction (also sometimes referred to as “labeling”) in step (C) is preferably effected or allowed to run for only 3 to 30 minutes at a temperature between 30 and 60° C., preferably for about 15 minutes (such as 12 to 18 minutes) at a temperature between 35 and 45° C., such as at about 40° C.

A further advantage of the present method is that it only comprises one radiochemical step (in which the radionuclide is involved). This is generally a benefit as it reduces unwanted (and unnecessary) losses of part of the prepared radioconjugates' activity due to the relatively short half-life of such radionuclides.

The chelation reaction mixture in step (C) preferably comprises a buffer or buffer system to control the pH. The buffer(s) in step (C) may be chosen among those known to be appropriate for the pH range of 7.1 to 10, such as 3-{[tris(hydroxylmethyl)methyl]amino}propanesulfonic acid (TAPS); N,N-bis(2-hydroxyethyl)glycine (Bicine); tris(hydroxymethyl)aminomethane (Tris, also referred to as tris(hydroxymethyl)methylamine); N-tris(hydroxymethyl)-methylglycine (Tricine), etc. In a particularly preferred embodiment, the chelation reaction mixture comprises tris(hydroxymethyl)aminomethane (Tris) as a buffer.

The “conjugated chelate compound” (also called “conjugate” herein) is a chelate compound conjugated (i.e. generally covalently linked) to a biological compound.

A “chelate compound” or “chelate” or “chelator” useful in the present invention are so-called bifunctional chelators which are compounds having the double functionality of sequestering metal ions combined to the ability to covalently bind a biological compound. Useful chelate compounds may thus be any appropriate chelating agent capable of reacting with a biological molecule, such as one or more selected from diethylene triamine pentaacetic acid (DTPA); ethylene diamine tetraacetic acid (EDTA); 1,4,7,10-tetraazacyclododecane-N,N′,N″,N′″-tetraacetic acid (DOTA); p-isothiocyanatobenzyl-1,4,7,10-tetraazacyclododecane-1,4,7,10-tetraacetic acid (p-SCN-Bz-DOTA); 1,4,7,10-tetraazacyclododecane-N,N′,N″-triacetic acid (DO3A); 1,4,7,10-tetraazacyclododecane-1,4,7,10-tetrakis(2-propionic acid) (DOTMA); 3,6,9-triaza-12-oxa-3,6,9-tricarboxymethylene-10-carboxy-13-phenyl-tridecanoic acid (B-19036); 1,4,7-triazacyclononane-N,N′,N″-triacetic acid (NOTA); 1,4,8,11-tetraazacyclotetradecane-N,N′,N″,N′″-tetraacetic acid (TETA); triethylene tetraamine hexaacetic acid (TTNA); trans-1,2-diaminohexane tetraacetic acid (CYDTA); 1,4,7,10-tetraazacyclododecane-1-(2-hydroxypropyl)-4,7,10-triacetic acid (HP-DO3A); trans-cyclohexane-diamine tetraacetic acid (CDTA); trans(1,2)-cyclohexane dietylene triamine pentaacetic acid (CDTPA); 1-oxa-4,7,10-triazacyclododecane-N,N′,N″-triacetic acid (OTTA); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); 2,2′,2″-(10-(2-(2,5-dioxopyrrolidin-1-yloxy)-2-oxoethyl)-1,4,7,10-tetraazacyclo-dodecane-1,4,7-triyl)triacetic acid (DOTA-NHS ester) and derivatives thereof.

A “biological compound” in the context of the present invention may be any appropriate naturally, synthetically, or recombinantly obtained or prepared compound selected from a protein, a peptide, an antibody or an antigen-binding fragment thereof, a protein comprising antigen-binding polypeptide sequences of an antibody, a monoclonal antibody, a fraction of a monoclonal antibody, such as a variable region thereof, a protein comprising an antigen binding sequence of an antibody, a polynucleotide, or any derivative of these compounds.

Thus, the term “radioconjugate” as used herein refers to chelate compound conjugated to a biological compound, wherein the chelate compound has been complexed with a radionuclide, such as Ac-225. The term “radioimmunoconjugate” more particularly refers to such a radioconjugate if the biological compound is a compound capable of using antibody-antigen bonding for the targeting described in the introductive part.

The conjugated chelate compound can be directly used in step (C). However, if necessary, the conjugated chelate compound may also be first prepared by reacting a chelate compound with a biological molecule in a preliminary conjugation step. Hence, in a further aspect, the method described herein preferably additionally comprises before said step (C) the following preparation step:
(A) conjugating a biological compound with a chelate compound in a conjugation reaction mixture to obtain a conjugated chelate compound.

This step may be done using any known method useful to link a chelator to a biological compound [e.g. Mirzadeh et al. 1990]. Furthermore, in this conjugation reaction, the chelate and the biological compounds may be any appropriate compounds, such as those already cited above. Preferably, the chelate compound is selected from DOTA and its functional derivatives. The biological compound is preferably a protein, a peptide, an antibody or a derivative thereof, particularly preferably a monoclonal antibody such as Lintuzumab (HuM195), Rituximab (trade names Rituxan® and MabThera®), Cetuximab (Erbitux®), Trastuzumab (Herceptin®), mAb2556 (anit-gp41) mAb c595 (anti-MUC1), anti-CD38-MAb MOR03087, MX35, F8 (specific to EDA fibronectin), L19 (specific to EDB fibronectin) and F16 (specific to domain A1 of tenascin C).

The conjugation reaction in step (A) is conducted for a time sufficient to obtain an adequate conjugation yield. The time necessary depends among others on the pair of reactants and the temperature at which the reaction takes place. In general, the reaction conditions useful for this step comprise reaction times from 30 minutes to 48 hours at temperatures between 15 and 40° C., preferably from 6 hours to 18 hours at temperatures between 25 and 35° C.

It is generally necessary or at least preferable to also control the pH of the conjugation mixture. A pH range which is useful for a particular conjugation reaction will depend among others on the pair of reactants; however, a particularly useful range of pH will be pH values from 7 to 10, more preferably 8 to 9.5 or even between 8.5 and 8.9. Appropriate buffers may be used to keep the pH in the selected range, such as those mentioned above or preferably bicarbonate or phosphate buffers.

If the method comprises a conjugation step (A), a purification of the conjugated chelate compound may be useful to eliminate unreacted chelate and biological compounds before proceeding further to the actual chelating step (C).

In a preferred embodiment of the method, the method thus further comprises between steps (A) and (C) the following step of:
(B) purifying the conjugated chelate compound obtained in step (A).

This step may be effected using any one or more of the known techniques, such as filtration, size exclusion chromatography, affinity purification, centrifugation, extraction, adsorption, dialysis, etc. A particularly preferred technique comprises ultrafiltration with a molecular weight cut-off of at least 10000 Da, more preferably of at least 20000 Da, even more preferably of at least 30000 Da. Depending on the compounds used in the conjugation step (A), the cut-off may even be at least 40000 Da or more.

Generally it will be desirable or even necessary to also adjust and control the pH during the purifying step (B). In such cases it may be advantageous to add a buffer or buffer system. A preferred buffer or buffer system is 4-(2-hydroxyethyl)-1-piperazineethanesulfonic acid (HEPES), bicarbonate or sodium acetate or combinations thereof. Preferably, the conjugation is performed at pH 8.5-8.9 in bicarbonate buffer (0.15 M NaCl/0.05 M NaHCO₃). This buffer may also be used in the first washing steps (e.g. 3×), then the product may be washed several times with the buffer system in which the final product is stored, e.g. 0.15 M NaCl/0.05 M Na-acetate, pH 7.2.

In a further aspect, further auxiliaries and additives may be added to the chelation reaction mixture if necessary or deemed useful. In one aspect, radioprotectants or stabilizers are further added to this chelation reaction mixture. Such radioprotectants or stabilizers may be chosen for example for povidone (polyvinylpyrrolidone, PVP), ascorbic acid, benzyl alcohol, cysteamine, cystamine, propylene glycol, dextran, and gentisic acid, preferably ascorbic acid or gentisic acid.

The radioprotectant(s) or stabilizer(s), preferably gentisic acid and/or ascorbic acid, is/are added in the chelation reaction mixture in step (C) either from the start before the actual chelation reaction begins, at any time during said chelation reaction or at the end of said chelation reaction. In a preferred aspect, the radioprotectant(s) or stabilizer(s) is/are added at the end of the chelation reaction (or in other words immediately after the reaction), i.e. generally after about 10 to 15 minutes.

In a still further aspect, the method further comprises after step (C) the following step:
(D) quenching the chelation reaction of step (C) by adding a quenching compound to the reaction mixture.

The quenching reaction, also referred to as termination reaction, may be useful to scavenge possibly unreacted (unchelated) radionuclide. This quenching or termination may be done by adding a quenching compound, such as a chelator. These chelators may be one or more of those cited above, however they need not to be bifunctional chelators, because they only need the functionality of sequestering metal ions, in particular Ac-225. One particularly appropriate quenching compound is for example diethylenetriaminepentaacetic acid (DTPA).

For any one or more of the steps described herein, it might be necessary to heat the corresponding reaction mixture. Such a heating may be performed using any conventional method or apparatus, such as a heating block or equivalent alternatives. Preferably, the heating is performed using microwave heating, especially for step (C).

### BRIEF DESCRIPTION OF THE DRAWINGS

Preferred embodiments of the invention will now be described, by way of example, with reference to the accompanying drawings in which:

FIG. 1 is a diagram showing the radiolabeling yield for synthesis of Ac-225-DOTA-rituximab according to the method according to the present invention (full squares) in comparison with the prior art described by Simon et al. (US 20120220754) (shown as empty circles);

FIG. 2 is a diagram showing the stability of ²²⁵Ac-DOTA-rituximab radioconjugate in human serum at 37° C. under 5% CO₂ atmosphere;

FIG. 3 is a diagram showing the scatchard analysis of the binding affinity of Ac-225-DOTA-rituximab to K422 lymphoma cells synthesized according to the method of the present invention.

Further details and advantages of the present invention will be apparent from the following detailed description of several not limiting examples and embodiments with reference to the attached drawings.

### EXAMPLES

Conjugation Reaction:

A solution of 0.34 mg of p-SCN-Bn-DOTA in 1 ml 0.15 M NaCl/0.05 M NaHCO3 (pH 8.5-8.9) is added to a reaction vial containing 5 mg of monoclonal antibody rituximab (anti-CD20) dissolved in 1 ml of 0.15 M NaCl/0.05 M NaHCO3 (pH 8.5-8.9). The mixture is stirred for 18 hours at 25° C. For removal of unconjugated p-SCN-Bn-DOTA chelate, the reaction mixture is subsequently filtered through an ultrafiltration unit with 30 kD cutoff (Amicon) until approximately 0.3 ml are left on top of the filter. Subsequently 1 ml of 0.15 M NaCl/0.05 M NaHCO3 (pH 8.5-8.9) is added to the filtration unit and passed through the filter until 0.3 ml of solution are remaining on top of the filter. This step is repeated three times. Subsequently 1 ml of 0.15 M NaCl/0.05 M sodium acetate (pH 7.2) is added to the filtration unit and passed through the filter until 0.3 ml of solution are remaining on top of the filter. This step is also repeated three times. Finally the purified conjugated monoclonal antibody (DOTA-rituximab) is taken up in 1.5 ml 0.15 M NaCl/0.05 M sodium acetate (pH 7.2).

Characterisation of the Conjugated Antibody:

The final concentration of the DOTA-rituximab conjugate in 0.15 M NaCl/0.05 M sodium acetate is analyzed by spectrophotometry or using a colourimetric method for protein assay. The ratio of DOTA-chelate molecules per molecule of monoclonal antibody is determined by spectrophotometry as described in [Dadachova E, Chappell L L and Brechbiel M.: “Spectrophotometric method for determination of bifunctional macrocyclic ligands in macrocyclic ligand-protein conjugates.” (Nucl Med Biol. 1999; 26(8):977-82)], by radiometric titration or mass spectrometry (e.g. MALDI-MS).

Radiolabelinq (Chelation) Procedure:

0.1 mg of DOTA-rituximab in 0.02 ml 0.15 M NaCl/0.05 M sodium acetate (pH 7.2) is added to a reaction vial containing 0.5 ml of 0.1 M TRIS buffer (pH 9.0). Subsequently 10 μl of Ac-225 stock solution in 0.1 M HCl containing 0.1 mCi to 0.5 mCi (3.7 MBq to 18.5 MBq) Ac-225 are added to the reaction vial. The reaction solution is mixed using a vortex mixer. An aliquot of 2 μl is withdrawn from reaction mixture and pipetted onto a pH paper to verify the pH is 8.5-9.0. If required, the pH is adjusted by addition of 0.1 M sodium hydroxide solution. Subsequently the solution is heated to 40° C. for 15 minutes.

Analysis of the Radiolabeling Yield Using Instant Thin Layer Chromatography (ITLC):

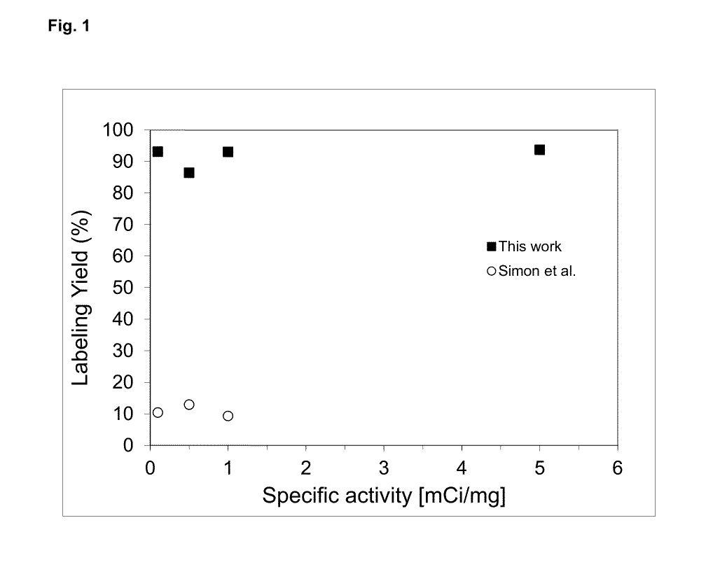

At the end of the chelation reaction, an aliquot of 1 μl of the reaction mixture is withdrawn for analysis of the radiochemical purity by instant thin layer chromatography (ITLC-SG, Agilent) using 0.05 M sodium citrate solution, pH 5.5 as solvent as described in [Essler M, Gärtner F C, Neff F, Blechert B, Senekowitsch-Schmidtke R, Bruchertseifer F, Morgenstern A, Seidl C. “Therapeutic efficacy and toxicity of (225)Ac-labelled vs. (213)Bi-labelled tumour-homing peptides in a preclinical mouse model of peritoneal carcinomatosis.” (Eur J Nucl Med Mol Imaging. 2012; 39(4):602-12)]. The radiolabeling yields obtained are typically well above 80%, often even above 90% as illustrated in FIG. 1 for specific activities of 0.1 to 5 mCi Ac-225 per mg monoclonal antibody.

As illustrated in FIG. 1, the radiolabeling yields obtained using the method described here are significantly higher than the radiolabeling yields obtained following the prior art described in Simon et al. (US2012/0220754 A1).

Optional: Purification of the Radioimmunoconjugate:

If deemed necessary, the radioimmunoconjugate can be purified by size exclusion chromatography using a PD10 column (Biorad). To this end, 0.01 ml of a solution containing 1.5 mg/ml DTPA and 0.05 ml of 20% ascorbic acid solution are added to the radiolabeling mixture (obtained after the radiolabeling procedure) and the entire mixture is loaded onto a PD10 column preconditioned with 10 ml 0.9% NaCl solution. Subsequently the column is washed with 2.36 ml of 0.9% NaCl solution. Discard the washings. Add 2 ml 0.9% NaCl solution on the column and collect the eluate containing the purified radioimmunoconjugate. The radiochemical purity of the purified radioimmunoconjugate typically exceeds 98%.

Stabilization:

In the absence of a suitable radioprotectant the radiochemical purity of the Ac-225 labeled radioimmunoconjugate gradually decreases with time due to radiolytic effects. In order to increase the stability of the radioimmunoconjugate, a radioprotectant is added. To this end, following the radiolabeling procedure step or following the optional purification step 1 ml of a 20% solution of ascorbic acid adjusted to pH 6 are added to the radioimmunoconjugate. However, the volume and the pH as well as the concentration of the ascorbic acid may vary. Furthermore, other radioprotectants may be used instead of the ascorbic acid.

Serum Stability of the Radioimmunoconjugate:

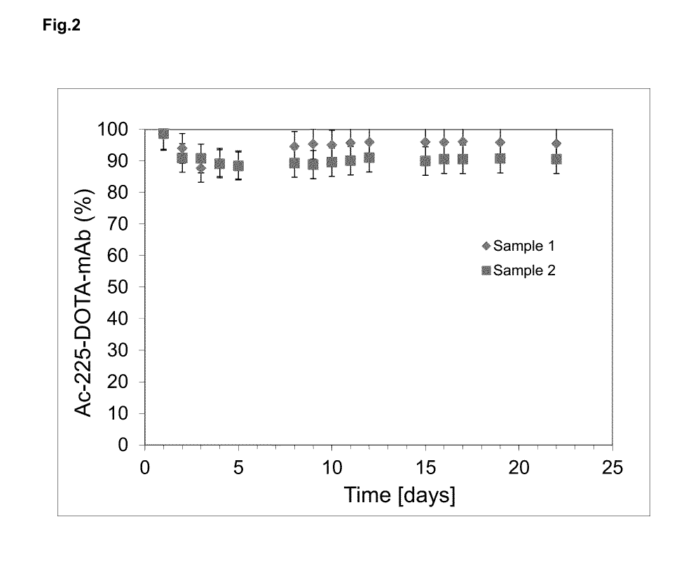

The stability of two samples of Ac-225 labeled DOTA-rituximab synthesized according to the method described above was studied in human serum. To this end an aliquot of 0.1 ml of purified radioimmunoconjugate was added to 1 ml of human serum and incubated at 37° C. under 5% CO₂ atmosphere. At various time points, an aliquot of the sample was analyzed by ITLC. The results are shown in FIG. 2. Both radioimminoconjugates show excellent stability over 22 days (exceeding two half-lives of Ac-225).

Binding Affinity of the Radioimmunoconjugate:

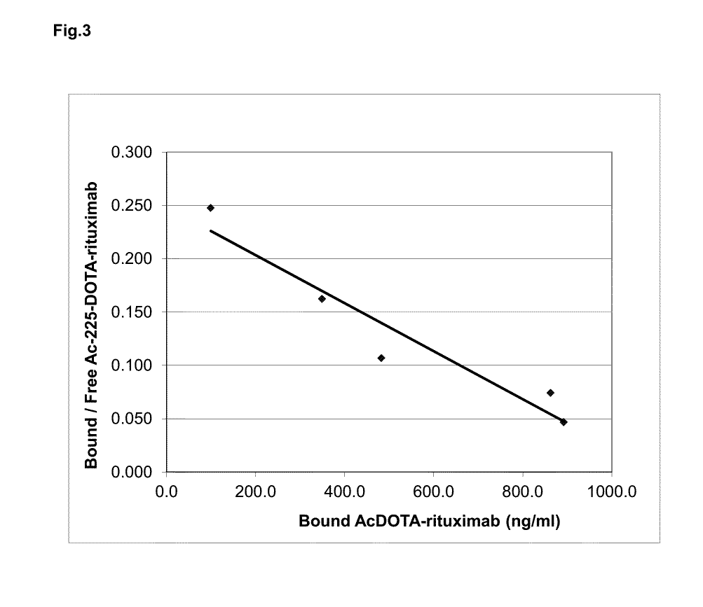

The binding affinity of an Ac-225-DOTA-rituximab radioimmunoconjugate synthesized according to the method disclosed here was investigated towards K422 lymphoma cells using a saturation binding assay as described in [Mario De Decker, Klaus Bacher, Hubert Thierens, Guido Slegers, Rudi A. Dierckx, Filip De Vos: “In vitro and in vivo evaluation of direct rhenium-188-labeled anti-CD52 monoclonal antibody alemtuzumab for radioimmunotherapy of B-cell chronic lymphocytic leukemia.” (Nuclear Medicine and Biology 35 (2008) 599-604)]. As shown in FIG. 3, the radioimmunoconjugate has preserved an excellent binding affinity with a value of 30 nM.

What is claimed is:

1. A method for producing a radioconjugate labeled with radionuclide Ac-225 comprising the following step:
(C) chelating radionuclide Ac-225 with a conjugated chelate compound in a chelation reaction mixture to obtain a radioconjugate labeled with Ac-225,
wherein the pH of the chelation reaction mixture is comprised between 7.1 and 10, preferably between 8.0 and 9.5, more preferably about 9.0.

2. The method according to claim 1, wherein the chelation reaction mixture in step (C) comprises tris(hydroxymethyl)aminomethane as a buffer.

3. The method according to claim 1, further comprising before step (C) the following step:
(A) conjugating a biological compound with a chelate in a conjugation reaction mixture to obtain a conjugated chelate compound,
wherein the chelate is selected from DOTA or derivatives thereof.

4. The method according to claim 3, further comprising between steps (A) and (C) the following step:
(B) purifying the conjugated chelate compound obtained in step (A).

5. The method according to claim 1, wherein the chelation reaction in step (C) is effected for 3 to 30 minutes at a temperature between 30 and 60° C. and preferably for about 15 minutes at a temperature between 35 and 45° C.

6. The method according to claim 1, wherein gentisic acid and/or ascorbic acid is added in the chelation reaction mixture in step (C) before the chelation reaction begins, during said chelation reaction or at the end of said chelation reaction, preferably at the end of the chelation reaction.

7. The method according to claim 1, further comprising after step (C) the following step:
(D) quenching the chelation reaction of step (C) by adding a quenching compound to the reaction mixture.

8. The method according to claim 1, wherein heating of the reaction mixture is performed using microwave heating.

9. The method according to claim 2, further comprising before step (C) the following step:
(A) conjugating a biological compound with a chelate in a conjugation reaction mixture to obtain a conjugated chelate compound,
wherein the chelate is selected from DOTA or derivatives thereof.

10. The method according to claim 9, further comprising between steps (A) and (C) the following step:
(B) purifying the conjugated chelate compound obtained in step (A).

11. The method according to claim 9, wherein the chelation reaction in step (C) is effected for 3 to 30 minutes at a temperature between 30 and 60° C. and preferably for about 15 minutes at a temperature between 35 and 45° C.

12. The method according to claim 9, wherein gentisic acid and/or ascorbic acid is added in the chelation reaction mixture in step (C) before the chelation reaction begins, during said chelation reaction or at the end of said chelation reaction, preferably at the end of the chelation reaction.

13. The method according to claim 9, further comprising after step (C) the following step:
(D) quenching the chelation reaction of step (C) by adding a quenching compound to the reaction mixture.

14. The method according to claim 9, wherein heating of the reaction mixture is performed using microwave heating.
